(19) United States
(12) Patent Application Publication
(10) Pub. No.: US 2024/0423743 A1
(43) Pub. Date: Dec. 26, 2024

(54) REUSEABLE STOWAGE COVER FOR ROBOTIC SURGICAL SYSTEM

(71) Applicant: Cilag GmbH International, Zug (CH)

(72) Inventors: Mary Margaret Scheunert, San Francisico, CA (US); Kandice Hoi-Ning Lau, San Francisico, CA (US); Clinton Denlinger, Cincinnati, OH (US)

(21) Appl. No.: 18/827,563

(22) Filed: Sep. 6, 2024

Related U.S. Application Data
(63) Continuation of application No. PCT/IB2023/052078, filed on Mar. 6, 2023.
(60) Provisional application No. 63/317,289, filed on Mar. 7, 2022.

(51) Int. Cl. A61B 46/10; A61B 34/30

(52) U.S. Cl. CPC A61B 46/10; A61B 34/30

(57) ABSTRACT

A robotic surgical system includes a surgical bed comprising a table configured to receive a patient, and a movable robotic arm coupled with the surgical bed and movable between a stowed configuration and a deployed configuration. A stowage cover including a flexible body is configured to selectively cover the movable robotic arm in the stowed configuration and is removable from the surgical bed to permit the movable robotic arm to transition from the stowed configuration to the deployed configuration. A sensor target is operatively coupled with the flexible body, and a sensor is configured to generate a signal in response to sensing a presence of the sensor target. A controller is configured to receive the signal from the sensor to determine that the stowage cover is positioned on the movable robotic arm in the stowed configuration.

### PRIORITY

This application is a continuation of International Patent Application No. PCT/IB2023/052078, filed Mar. 6, 2023, entitled “Reusable Stowage Cover for Robotic Surgical System,” which claims priority to U.S. Provisional Patent Application No. 63/317,289, entitled “Reusable Stowage Cover for Robotic Surgical System,” filed Mar. 7, 2022, the disclosures of each of which are incorporated by reference herein, in their entirety.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, holding or driving a needle, grasping a blood vessel, dissecting tissue, or cauterizing tissue.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of a table-based robotic system configured for a laparoscopic procedure;

FIG. 2 depicts a perspective view of a second example of a table-based robotic system configured to stow one or more robotic arms;

FIG. 3 depicts a perspective view of a third example of a table-based robotic system;

FIG. 4 depicts an end elevational view of the table-based robotic system of FIG. 3;

FIG. 5 depicts an end elevational view of the table-based robotic system of FIG. 4 including a pair of robotic arms;

FIG. 6 depicts a partially exploded perspective view of one of the robotic arms of FIG. 4 with an instrument driver and a surgical instrument;

FIG. 7A depicts a side elevational view of another example of a table-based robotic system that includes a stowage cover, where robotic arms of the system are in a stowed configuration and adjustable arm supports are in a lower position, with the stowage cover being shown in phantom;

FIG. 7B depicts a side elevational view of the table-based robotic system of FIG. 7A, but with the adjustable arm supports being moved to a higher position, with the stowage cover being shown in phantom;

FIG. 8 depicts an enlarged perspective view of a portion of the table-based robotic system of FIG. 7A, with a first flexible body of the stowage cover covering a first portion of the system and a second flexible body of the stowage cover shown in phantom covering a second portion of the system;

FIG. 9 depicts an enlarged side elevational view of a portion of the table-based robotic system of FIG. 7A that includes the first and second flexible bodies;

FIG. 10 depicts an enlarged end elevational view of a portion of a first bar of the table-based robotic system of FIG. 7A;

FIG. 11 depicts an enlarged end elevational view of a portion of an adjustable arm support of the table-based robotic system of FIG. 7A and the stowage cover of FIG. 7A;

FIG. 12 depicts a diagrammatic view of an example of a sensing assembly for use with the table-based robotic system of FIG. 7A; and

FIG. 13 depicts a diagrammatic view of an example of a method of operating the table-based robotic system of FIG. 7A.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “upper” and lower” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

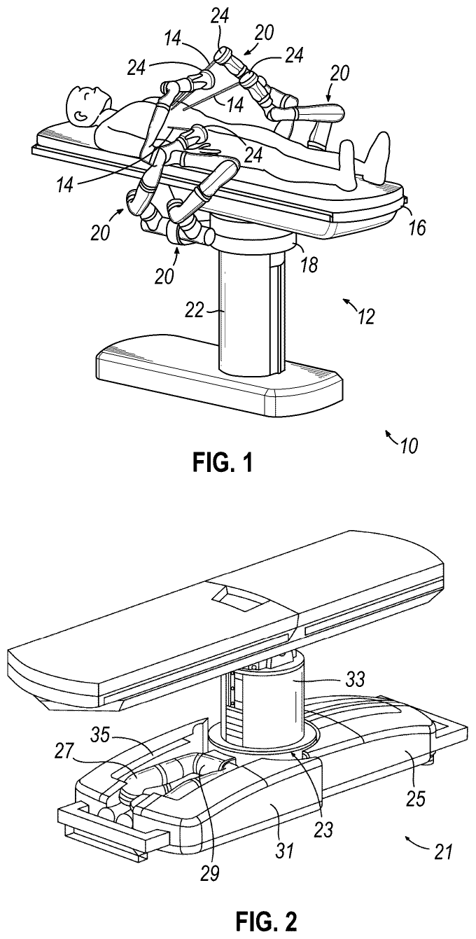

FIG. 1 shows an example of a robotically-enabled medical system, including a first example of a table-based robotic system (10). Table-based robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of table-based robotic system (10) may be constructed and operable in accordance with at least some of the teachings of any of the various patents, patent application publications, and patent applications that are cited herein.

A. Example of Table-Based Robotic System with Annular Carriage

With respect to FIG. 1, table-based robotic system (10) includes table system (12) having a platform, such as a table (16), with a plurality of carriages (18) which may also be referred to herein as “arm supports,” respectively supporting the deployment of a plurality of robotic arms (20). Table-based robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, table-based robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Table-based robotic system (10) may also include a tower (not shown) that divides the functionality of table-based robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

B. Example of Table-Based Robotic System with Integral Arm Covers

As noted above, table-based robotic system (10) may stow robotic arms (20) under table (16) when robotic arms (20) are not in use. In some versions, a table-based robotic system may provide some kind of covering over stowed robotic arms that are not in use. FIG. 2 illustrates an example of such a table-based robotic system (21) that is operable to stow and cover robotic arms (27). In table-based robotic system (21) of this example, robotic arms (27) are supported by carriages (23) via arm mounts (29). Carriages (23) may be vertically translated along a column (33). As shown, carriages (23) may be vertically translated downwardly into base (25) to stow robotic arms (27), arm mounts (29), and carriages (23) within base (25).

Table-based robotic system (21) of this example further includes base cover panels (31) as part of base (25). Base cover panels (31) are thus an integral component of table-based robotic system (21) in this example. Base cover panels (31) may be moved between an open configuration (e.g., as shown in the left-hand region of FIG. 2) and a closed configuration (e.g., as shown in the right-hand region of FIG. 2). When base cover panels (31) are in the open configuration, base cover panels (31) allow robotic arms (27), arm mounts (29), and carriages (23) to translate vertically into and out of the lowered, stowed position. When base cover panels (31) are in the closed configuration, base cover panels (31) may cover robotic arms (27), arm mounts (29), and carriages (23) while robotic arms (27), arm mounts (29), and carriages (23) remain in the lowered, stowed position. When base cover panels (31) are in the closed configuration, base covers (310) may protect the stowed robotic arms (27), arm mounts (29), and carriages (23). Each base cover panel (31) further includes a membrane (35) along the edges of its opening, which may prevent dirt and fluid ingress when base cover panels (31) are in the closed configuration. In some versions, base cover panels (31) are driven manually; while in other versions, base cover panels (31) are driven automatically.

C. Example of Table-Based Robotic System with Bar Carriage

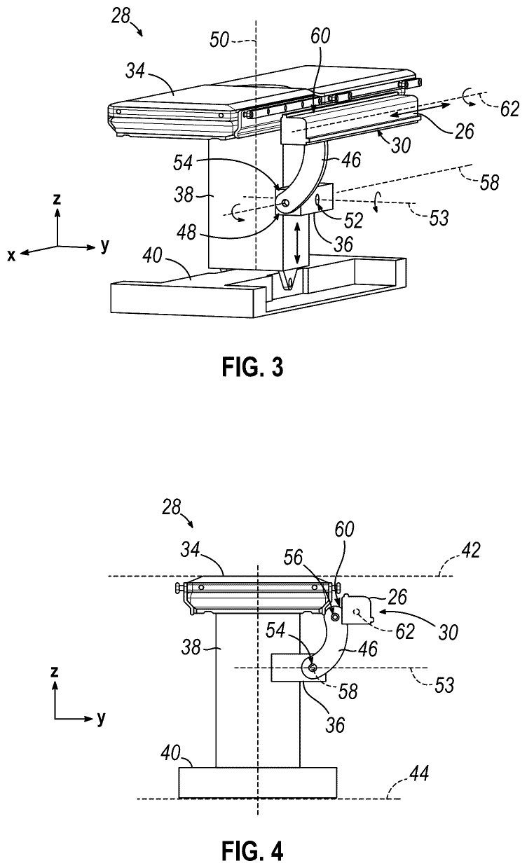

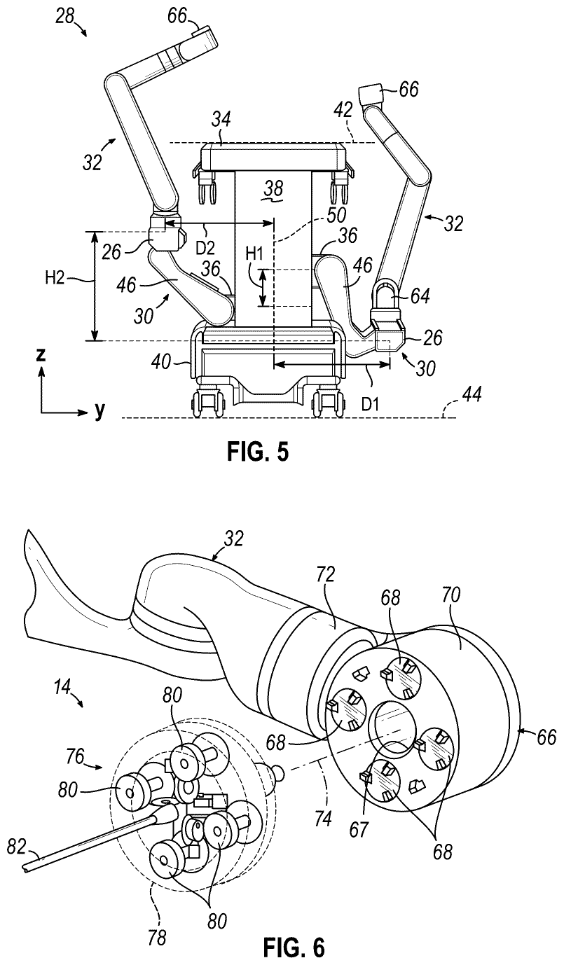

FIGS. 3-5 show another example of a table-based robotic system (28). Table-based robotic system (28) of this example includes one or more adjustable arm supports (30) including bars (26) that are configured to support one or more robotic arms (32) relative to a table (34). In the present example, a single adjustable arm support (30) (FIGS. 3-4) and a pair of adjustable arm supports (30) (FIG. 5) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to table-based robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 3-5, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) may maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 5 shows a version of table-based robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 5, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

FIG. 6 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

As also shown in FIG. 6, surgical instrument (14) includes an elongated shaft assembly (82) and an instrument base (76) with an attachment interface (78) having a plurality of drive inputs (80) configured to respectively couple with corresponding drive outputs (68). Shaft assembly (82) of instrument (14) extends from a center of instrument base (76) with an axis substantially parallel to the axes of the drive inputs (80) as discussed briefly above. With shaft assembly (82) positioned at the center of instrument base (76), shaft assembly (82) is coaxial with instrument driver axis (74) when attached and movably received in clearance bore (67). Thus, rotation of rotational assembly (70) causes shaft assembly (82) of surgical instrument (14) to rotate about its own longitudinal axis while clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

While not shown in FIGS. 3-5, table-based robotic system (28) may be configured to provide stowage of robotic arms (32) under table (34) when robotic arms (32) are not in use. In some such versions, base (40) may include an integral cover feature like base cover panels (31) to cover robotic arms (32) and one or more features of arm support (30) when robotic arms (32) are stowed. In some other versions, a removable cover may be used to cover robotic arms (32) and one or more features of arm support (30) when robotic arms (32) are stowed.

### II. Example of Table-Based Robotic System and Method with Removable and Reusable Cover

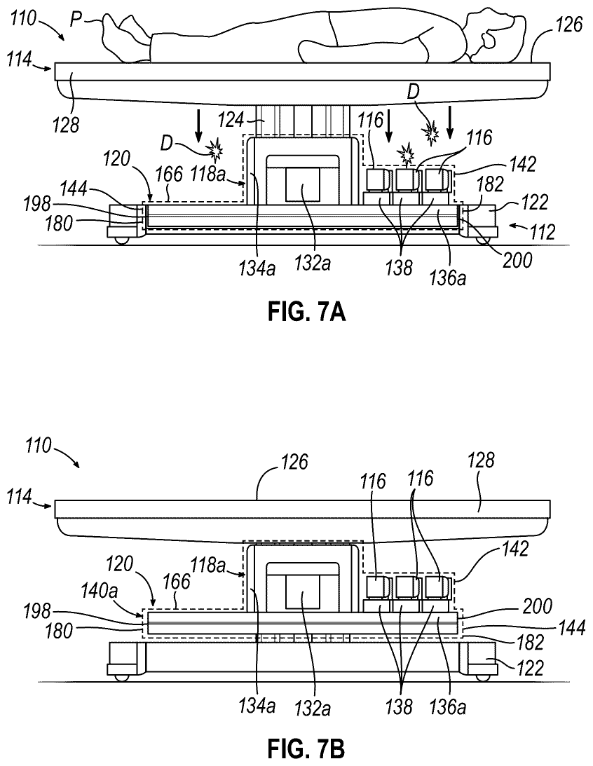

As noted above, different kinds of table-based robotic systems (10, 21, 28) may allow robotic arms (20, 27, 32) to be stowed under table (16, 34) when robotic arms (20, 27, 32) are not in use. By way of example only, such scenarios may occur in between robotic surgical procedures and/or during manual laparoscopy procedures. During manual laparoscopy procedures, debris (D) (see FIG. 7A) may tend to fall from table (16, 34) onto lower portions of table-based robotic systems (10, 21, 28). Debris (D) may include solids, and/or liquids (e.g., bodily fluid and blood). For example, this debris (D) may drop down onto robotic arms (20, 27, 32), column (22), base (25, 40), carriages (18, 23) and/or adjustable arm support (30). For table-based robotic systems (10, 21, 28), this debris (D) may contribute to degradation of the capital equipment, provide more difficult cleaning procedures, and/or present a potential risk of cross-contamination between patients. Additionally, lower portions of table-based robotic systems (10, 21, 28) may get wet when fluid splashes up (e.g., when a user mops or otherwise cleans the floor) in between robotic surgical procedures.

As a result, it may be beneficial to protect table-based robotic systems (10, 21, 28) from soiling or damage due to debris (D) falling from above and/or fluid splashing up from below. Additionally, it may be beneficial to cover at least some portions of table-based robotic systems (10, 21, 28) to prevent this contamination from occurring in between procedures with different patients (P) and at any time during port placement or manual laparoscopy. While at least a portion of each robotic arm (20, 27, 32) may be non-sterile during a surgical procedure, it may still be desirable to keep components of table-based robotic systems (10, 21, 28) clean from debris (D), including robotic arms (20, 27, 32).

To the extent that base cover panels (31) may provide some degree of coverage and protection in the context of table-based robotic system (21), it may be desirable to instead provide a cover that is removable rather than having the cover be an integral component of the table-based robotic system. In some cases, a removable cover may be easier to clean and/or replace as needed. A removable cover may also minimize the cost and/or complexity of the table-based-robotic system.

While a disposable single-use cover may protect table-based robotic systems (10, 21, 28) from liquids, a single-use cover may need to be replaced once soiled or removed (even if not soiled). This may create additional work for the user, as the user would remove the single-use cover, dispose of the single-use cover, and apply a new single-use cover. A reusable stowage cover may provide cleaning simplification, as the user would not need to remove the single-use cover, clean the equipment underneath, and re-install a new single-use cover.

Moreover, in some medical facilities, untrained users are not permitted to directly touch capital equipment (e.g., table-based robotic systems (10, 21, 28) and integral components thereof). In such facilities, a reusable stowage cover may allow these non-robotically trained users to perform their jobs without deviation. For example, hospital cleaning protocols may prevent non-robotically trained users from replacing disposable single-use covers from table-based robotic system (10, 21, 28). A reusable stowage cover may allow non-robotically trained users to simply wipe down the outer surface of the stowage cover, thus treating it like an operating room hospital bed.

A single-use cover might also tend to not perform as well as a reusable cover in protecting the capital equipment; and may not fit as snugly as a reusable stowage cover. Additionally, single-use covers may tend to be less environmentally friendly than a reusable cover, as two single-use covers may be used in between each procedure in some scenarios (e.g., one single-use cover per bar (26)). Similarly, single-use covers may add to the waste burden a hospital produces. Single-use covers may also tend to be costly (when considered cumulatively). To the extent that a hard-shell case may be used to cover the capital equipment, it may be difficult to suitably store the hard-shell case within the operating room when not in use.

An example of a removable and reusable cover that may provide the protection described above while also providing potential benefits of removability, reusability, and storability is described in greater detail below.

### A. Example of Removable and Reusable Cover for Table-Based Robotic System

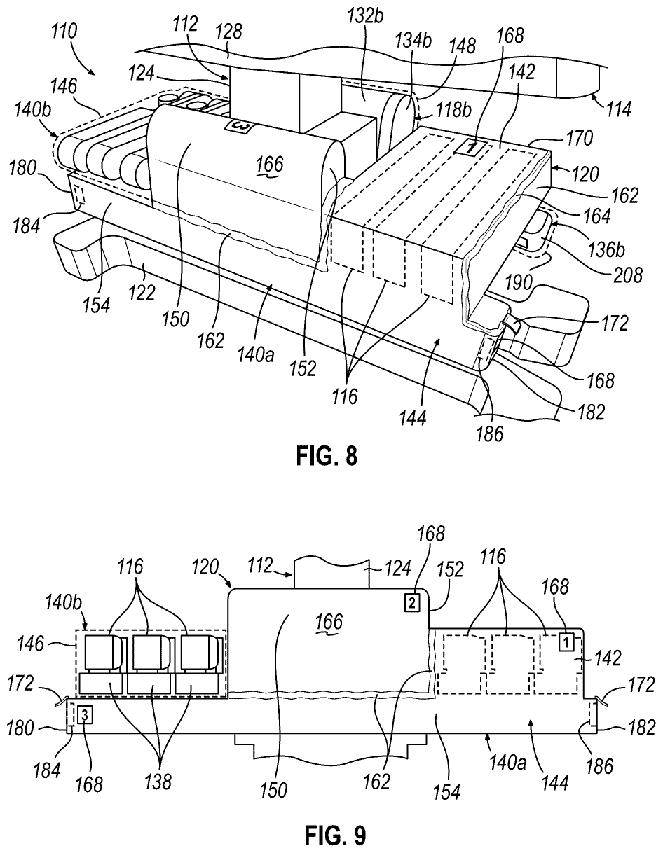

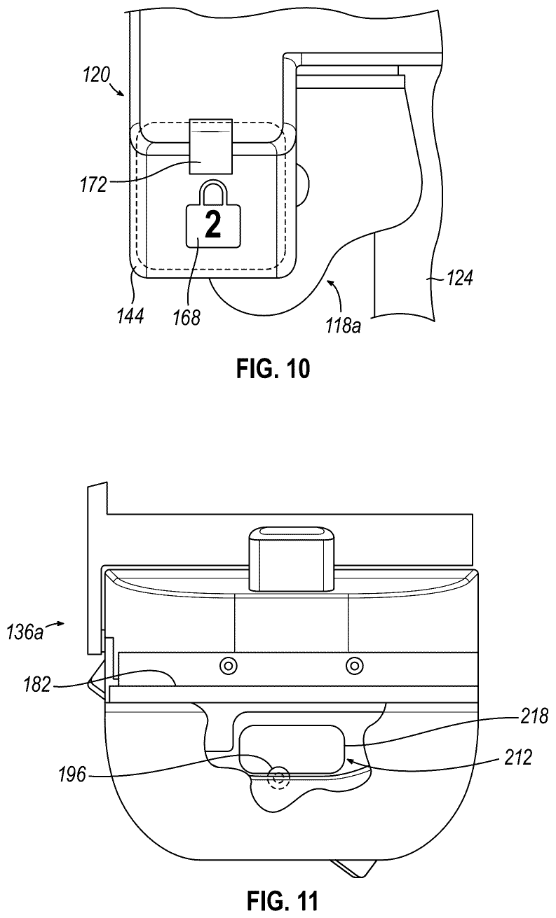

FIGS. 7A-10 show an example of a table-based robotic system (110). As shown, table-based robotic system (110) includes a support structure (112), a surgical bed (114), a plurality of robotic arms (116), at least one adjustable arm support (shown as first and second adjustable arm supports (118a-b)), and a stowage cover (120). As shown in FIGS. 7A-8, support structure (112) includes a base (122) and a column (124) to support surgical bed (114). Surgical bed (114) includes a table (126) configured to receive a patient (P). Table (126) may be configured to tilt to a desired angle during use, such as during laparoscopic procedures. Surgical bed (114) defines a first lateral side (128) and a second lateral side (not shown) that is disposed opposite first lateral side (128).

Robotic arms (116) may be configured and operable like robotic arms (32) described above. Robotic arms (116) may be independently movable relative to one another. Robotic arms (116) are operable to transition between a stowed configuration (see FIGS. 7A-9) and a deployed configuration (see FIG. 5 regarding robotic arms (32)). In the stowed configuration, robotic arms (116) are positioned under table (126) of surgical bed (114). In the deployed configuration, robotic arms (116) are configured to move relative to surgical bed (114) (e.g., for use in a robotic surgical procedure on patient (P)). While three robotic arms (116) are shown in FIGS. 7A-7B that are mounted at first lateral side (128) of surgical bed (114), more or fewer robotic arms (116) may be mounted at first lateral side (128) of surgical bed (114). In the example shown in FIGS. 8-9, table-based robotic system (110) includes a total of six robotic arms, with three robotic arms (116) mounted on first lateral side (128) and three robotic arms (116) mounted on the second lateral side of surgical bed (114).

First and second adjustable arm supports (118a-b) may be configured and operable like adjustable arm supports (30). First adjustable arm support (118a) is mounted on first lateral side (128), and second adjustable arm support (118b) is mounted on the second lateral side (not shown). First and second adjustable arm supports (118a-b) may move independently of one another. As shown in FIGS. 7A-7B, first adjustable arm support (118a) includes a first vertical carriage (132a), a first bar connector (134a), and a first bar (136a). Similarly, as shown in FIG. 8, second adjustable arm support (118b) includes a second vertical carriage (132b), a second bar connector (132b), and a second bar (136b). Connecting portions (138) of respective robotic arms (116) couple with either first bar (136a) or second bar (136b). It is also envisioned that adjustable arm supports (118a-b) may include a variety of other suitable support structures including but not limited to ring shaped carriages (e.g., similar to carriages (18) described above). While two adjustable arm supports (118a-b) are shown, more or fewer adjustable arm supports (118a-b) may be provided. As shown in FIG. 8, first and second bars (136a) extend parallel to table (126); however, table (126) may be tilted as described above.

Adjustable arm supports (118a-b) are configured to transition at least between a lower position (see FIG. 7A) and an upper position (see FIG. 7B). The lower position may be referred to as a “low stow” position. Similarly, the upper position may be referred to as “high stow” position. An intermediate position, also referred to as an “intermediate stow” position, may be located between the upper, “high stow” position and the lower, “low stow” position. In some versions, the intermediate stow position may be used when transporting table-based robotic system (110) between operating rooms, the high stow position may be used when a user is cleaning a floor around table-based robotic system (110), and the low stow position may be used in other scenarios.

Stowage cover (120) is configured to cover at least a portion of the table-based robotic system (110). For example, stowage cover (120) may selectively cover at least one robotic arm (116) and at least a portion of at least one adjustable arm support (118a-b). Stowage cover (120) is configured to enable a full robotic range of motion of surgical bed (114) while stowage cover (120) remains actively covering the desired portion of table-based robotic system (110). For example, stowage cover (120) does not prevent table (126) of surgical bed (114) from tilting or lowering. Particularly, first and second bars (136a-b) of adjustable arm support (118) and robotic arms (116) may move between the lower and upper positions without disturbing or otherwise affecting stowage cover (120).

Stowage cover (120) may include at least one flexible body. As shown in FIGS. 8-9, stowage cover (120) of the present example includes first and second flexible bodies (140a-b). Second flexible body (140b) is shown in phantom in FIGS. 8-9. While first and second flexible bodies (140a-b) are shown as being entirely separable from one another, first and second flexible bodies (140a-b) may be at least partially coupled together in some versions. First and second flexible bodies (140a-b) may work in combination to cover each adjustable arm support (118a-b) and each robotic arm (116). While first and second flexible bodies (140a-b) are described as being used in combination with each other below, it is envisioned that first and second flexible bodies (140a-b) may be used individually if a portion of robotic arms (116) is intended to be left uncovered.

First flexible body (140a) may cover first adjustable arm support (118a) and a portion of robotic arms (116) that are coupled with first adjustable arm support (118a). Similarly, second flexible body (140b) may cover second adjustable arm support (118b) and a portion of robotic arms (116) that are coupled with second adjustable arm support (118b). In some versions, first and second flexible bodies (140a-b) are configured to simultaneously cover robotic arms (116) in the stowed configuration and first and second bars (136a-b) in the upper position, in the lower position, and during a transition between the upper position and the lower position. Particularly, first and second flexible bodies (140a-b) may collectively cover first and second bars (136a-b) and robotic arms (116) and in the upper position, in the lower position, and during the transition between the upper position and the lower position. It is also envisioned that a single flexible body (not shown) may cover each adjustable arm support (118a-b) and each robotic arm (116). First and second flexible bodies (140a-b) are configured to closely conform to robotic arms (116).

First and second flexible bodies (140a-b) each define a plurality of compartments. For example, first flexible body (140a) includes first and second compartments (142, 144). Similarly, as shown in in FIG. 8, second flexible body (140b) includes first and second compartments (146, 148). As shown, first compartments (142, 146) are configured to simultaneously cover respective sets of robotic arms (116) in the stowed configuration. Second compartments (144, 148) are configured to cover respective adjustable arm supports (118a-b) in the stowed configuration. For example, second compartment (144) of first flexible body (140a) may include a first portion (150) to receive first vertical carriage (132a), a second portion (152) to receive first bar connector (134a), and a third portion (154) to receive first bar (136a). Similarly, second compartment (148) of second flexible body (140b) may include a first portion to receive first vertical carriage (132b), a second portion to receive first bar connector (134b), and a third portion to receive second bar (136b).

Compartments (142, 144) of first flexible body (140a) may be joined together at seams (162). While not shown, compartments (146, 148) of second flexible body (140b) may be joined together at seams. In some versions, seams (162) may be sufficient to keep first and second flexible bodies (140a-b) of stowage cover (120) in place without additional coupling features. Seams (162) may ensure that stowage cover (120) generally retains the shape when removed from table-based robotic system (110) and folded off-robot; or when placed on table-based robotic system (110). Seams (162) may be formed using a variety of methods including, but not limited to, ultrasonic welding and heat sealing. In some versions, seams (162) may be covered with seam tape (164). The snug fit of first and second flexible bodies (118a-b) may prevent first and second flexible bodies (140a-b) from wrinkling to prevent the formation of crevices or cavities in which debris (D) may accumulate.

First and second flexible bodies (140a-b) may be formed using a liquid-impermeable material. The liquid-impermeable material may allow a user to wipe an outer surface (166) of first and second flexible bodies (118a-b) down without removing first and second flexible bodies (140a-b) from table-based robotic system (110). First and second flexible bodies (140a-b) of stowage cover (120) may withstand gross contaminants from above and below and enable cleaning and disinfecting. In other words, stowage cover (120) may allow for repeated removal and installation; and multiple cycles of cleanings when soiled using operating room chemicals. One such suitable flexible fabric may include Sure-Chek® fabric, commercially available from Herculite, Inc. of Emigsville, Pennsylvania. Alternatively, any other suitable material or combination of materials may be used.

Stowage cover (120) may have an opaque, semi-opaque, semitransparent, or transparent material to allow for logos, instructions for use, and/or warnings to be printed on stowage cover (120). For example, stowage cover (120) may include indicia (168) indicating surfaces to facilitate a user in positioning stowage cover (120) over robotic arms (116). Indicia (168) may be positioned on each of compartments (142, 144, 146, 148). For example, indicia (168) disposed on first compartments (142, 146) may include a graphic or text indicating robotic arms (116) are disposed underneath. Patterns of indicia (168) may aid the user in differentiating between different faces of stowage cover (120) as the user prepares to install stowage cover (120). In some versions, pill-shapes may define the edge of the wrapped graphic. Subtle directionality of indicia (168) may reinforce the user's intuitive sense of how to apply and remove stowage cover (120).

First and second flexible bodies (140a-b) of stowage cover (120) may be completely flexible or include select portions that are generally rigid. For example, stowage cover (120) may include at least one stiffening member (170) that is configured to provide rigidity to a predetermined portion of stowage cover (120). Stiffening members (170), also referred to as spines, may provide rigidity of select portions of first and second flexible bodies (140a-b). Stiffening members (170) be integrated along seams (162) and/or along areas that are generally planar to case installation (e.g., along horizontal regions). Stiffening members (170) may include flat members and/or tubular members, such as strips or rods, etc. Stiffening members (170) may be formed of a generally rigid, semi-rigid, or resilient material such as metal or plastic. Stiffening members (170) and/or seams (162) may provide a structured shape and size for stowage cover (120) to match the shape of the grouping of robotic arms (116) in the stowed configuration. Stowage cover (120) may include one or more optional pull tabs (172) configured to assist a user in positioning stowage cover (120) relative to robotic arms (116) and first and second bars (136a-b). Pull tabs (172) may allow for quicker insertion and removal of stowage cover (120).

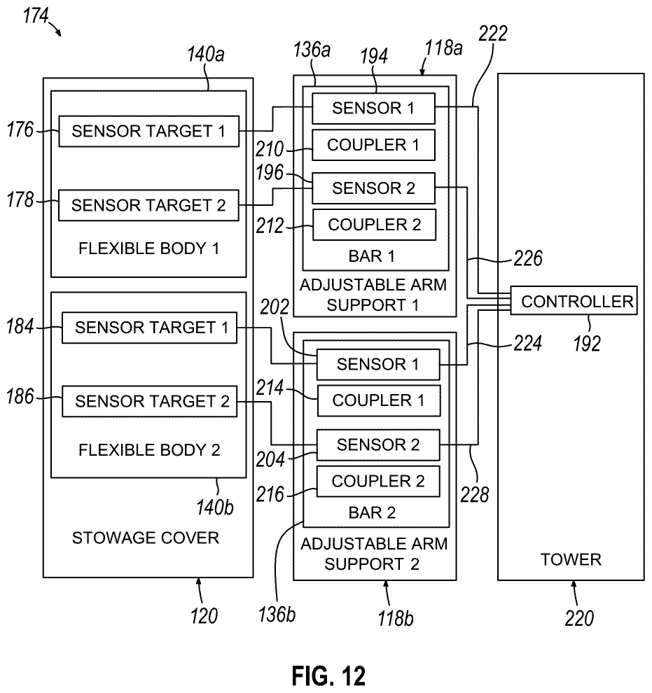

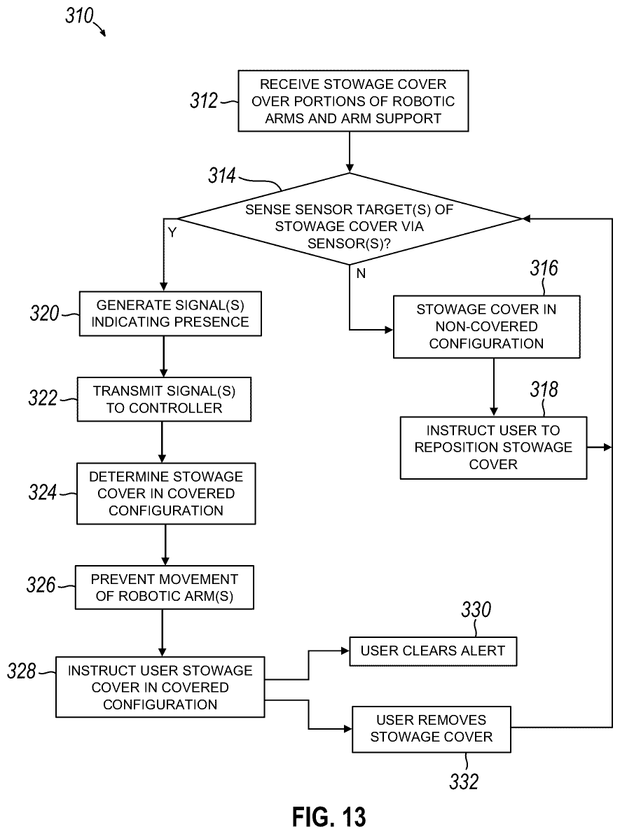

While not shown, table-based robotic system (110) may include a receptacle or other feature to provide storage of stowage cover (120) when stowage cover (120) is removed from robotic arms (160) and arm supports (118a-b). Alternatively, stowage cover (120) may be stored on a separate cart or storage bin located in a tower (220), as described below in reference to FIG. 12. Some versions of stowage cover (120) may be folded and placed in such a receptacle during a robotic surgical procedure, such that stowage cover (120) may be readily accessed and placed over robotic arms (160) and arm supports (118a-b) after the robotic surgical procedure is completed and robotic arms (160) and arm supports (118a-b) have been suitably cleaned.

While stowage cover (120) is described above in the context of table-based robotic system (110), stowage cover (120) may be readily modified for use with other kinds of table-based robotic systems, including but not limited to table-based robotic systems (10, 21, 28) described above. The functionality and utility of stowage cover (120) described herein is thus not limited to the specific context of table-based robotic system (110).

### B. Example of Sensing of Stowage Cover

In some versions, table-based robotic system (110) includes additional components configured to determine the status of stowage cover (120). This sensing may determine whether or not stowage cover (120) is disposed over robotic arms (116) and/or adjustable arm supports (118a-b). To that end, as shown in FIG. 12, system (110) may optionally include a sensing assembly (174). In some scenarios, sensing assembly (174) may prevent manual laparoscopy procedures from starting without stowage cover (120) covering the desired portion of table-based robotic system (110); or prevent a robotic surgical procedure from starting with stowage cover (120) covering the portion of table-based robotic system (110). Sensing assembly (174) may prevent a user from misplacing stowage cover (120) or from forgetting to place stowage cover (120) back on table-based robotic system (110) after a robotic procedure.

In some versions, stowage cover (120) includes at least one sensor target. In the present example, stowage cover (120) includes a plurality of sensor targets detectable by table-based robotic system (110) when table-based robotic system (110) is powered on. As shown in FIG. 12, first flexible body (140a) includes first and second sensor targets (176, 178). First flexible body (140a) includes opposing first and second ends (180, 182). First sensor target (176) is operatively coupled with a first end (180) of first flexible body (140a) and second sensor target (178) is operatively coupled with second end (182) of first flexible body (140a). Similarly, second flexible body (140b) includes first and second sensor targets (184, 186). Second flexible body (140b) includes a first end (not shown) and a second end (190). First sensor target (184) is operatively coupled with the first end of second flexible body (140b) and second sensor target (186) is operatively coupled with second end (190) of second flexible body (140b). Magnets, zippers, and/or other fastening structures may be incorporated into first and second flexible bodies (140a-b) of stowage cover (120) to fasten stowage cover (120) to itself and with select portions of table-based robotic system (110). Magnets may allow for hermetic seals to be maintained.

Table-based robotic system (110) may include at least one sensor and a controller (192), which may be located in a tower (220). As shown, sensing assembly (174) includes first and second sensors (194, 196) disposed on opposing first and second ends (198, 200) of first bar (136a) and first and second sensors (202, 204) disposed on opposing ends (208) of second bar (136b). Sensors (194, 196) may be located at first and second ends (198, 200) of first bar (136a) to ensure first flexible body (140a) is correctly positioned. Similarly, sensors (202, 204) may be located at ends (208) of second bar (136b) to ensure second flexible body (140b) is correctly positioned. First and second ends (198, 200, 208) of first and second bars (136a-b) may be the final portions to be covered by first and second flexible bodies (140a-b) of stowage cover (120). First sensor (194) of first bar (136a) is configured to generate a first signal (222) in response to first sensor (194) sensing a presence of first sensor target (176) of stowage cover (120). Similarly, first sensor (202) of second bar (136b) is configured to generate a first signal (224) in response to first sensor (202) sensing a presence of first sensor target (184) of stowage cover (120).

In some versions, first sensor (194, 202) includes a proximity sensor, and first sensor target (176, 184) includes a proximity sensor target configured to be sensed by proximity sensor. For example, first sensor (194, 202) may include a Hall effect sensor configured to respond to presence of a magnet. Similarly, second sensor (196, 204) is configured to sense a presence of second sensor target (178, 186). In some versions, second sensor (196, 204) includes a proximity sensor, and second sensor target (178, 186) includes a proximity sensor target configured to be sensed by proximity sensor. For example, second sensor (196, 204) may include a Hall effect sensor configured to respond to presence of a magnet. A variety of other sensors may be incorporated including optical sensors, etc.

In some versions, first and second adjustable arm supports (118-b) may include at least one coupling feature configured to couple with a coupling feature of stowage cover (120) to couple stowage cover (120) with adjustable arm support (118) and/or robotic arms (116). For example, coupling features (210, 212) may be located at first and second ends (198, 200) of first bar (136a) to ensure first flexible body (140a) is properly positioned. Similarly, coupling features (214, 216) may be located at first end and second end (208) of second bar (136b) to ensure second flexible body (140b) is properly positioned. As shown in FIG. 11, coupling features (210, 212, 214, 216) may include a least one ferromagnetic feature (shown as a ferromagnetic plate (218) in FIG. 11) to magnetically couple with sensor targets (176, 178, 184, 186) to couple first and second flexible bodies (140a-b) with respective first and second bars (136a-b).

Controller (192) is configured to receive first signal (222, 224) from first sensor (194, 202) to determine that stowage cover (120) is positioned over robotic arms (116). Controller (192) is configured to prevent movement of robotic arms (116) in response to determining that stowage cover (120) is in the covered configuration. For example, controller (192) is configured to determine that first and second flexible bodies (140a-b) of stowage cover (120) are positioned on robotic arms (116) based on a combination of the first sensor (194, 202) sensing the presence of the first sensor target (176, 184) and second sensor (196, 204) sensing the presence of the second sensor target (178, 186). Controller (192) is configured to provide an alert to a user in response to either first sensor (194, 202) not sensing the presence of first sensor target (176, 184) or second sensor (196, 204) not sensing the presence of second sensor target (178, 186). In the event that the operator enters a command via a user input device (e.g., joystick, exoskeletal glove, master manipulator, etc.) to drive movement of a robotic arm (116) when controller (192) receives signals from sensors (194, 196, 202, 204) that stowage cover (120) is in the covered configuration, controller (192) may respond with an alert and otherwise prevent any robotic arms (116) from moving in response to the operator's command until stowage cover (120) has been properly removed.

### C. Example of Method of Operation

FIG. 12 depicts an example of a method (310) of using stowage cover (120) in table-based robotic system (110). Method (310) begins after a robotic surgical procedure using robotic arms (116) has been completed, after robotic arms (116) have been cleaned as needed, and after robotic arms (116) have been moved from the deployed configuration to the stowed configuration. At step (312), method (310) includes receiving stowage cover (120) over robotic arms (116) when robotic arms (116) are in the stowed configuration. For example, one or more users may manually position stowage cover (120) over table-based robotic system (110). First compartments (142, 146) of first and second flexible bodies (140a-b) may receive robotic arms (116) when robotic arms (116) are under table (126) of table-based robotic system (110) in the stowed configuration. Second compartments (144, 148) of first and second flexible bodies (140a-b) may receive first and second adjustable arm supports (118a-b) as described above. It is envisioned that first and second flexible bodies (140a-b) of stowage cover (120) may be applied when arm supports (118a-b) are in the upper position, when arm supports (118a-b) are in the intermediate position, or when arm supports (118a-b) are in the lower position.

At step (314), method (310) includes sensing for the presence of any applicable sensor targets (176, 178, 184, 188) via any applicable corresponding sensors (194, 196, 202, 204). This step (314) may be executed via controller (192) based on signals (222, 224, 226, 228) (or lack of signals (222, 224, 226, 228)) from sensors (194, 196, 202, 204).

In scenarios where applicable sensor targets (176, 178, 184, 188) are not sensed at step (314), method (310) proceeds to step (316). At step (316), method (310) includes determining, via controller (192), that stowage cover (120) is not in a fully covering configuration based on absence of signals (222, 224, 226, 228) from sensors (194, 196, 202, 204). In some versions, lack of both first and second signals (222, 226) for first flexibly body (140a) indicates improper placement of first flexibly body (140a). Similarly, lack of both first and second signals (224, 228) for second flexibly body (140b) indicates improper placement of second flexibly body (140b). When the user incorrectly places stowage cover (120) on table-based robotic system (110), sensors (194, 196, 202, 204) in first and second bars (136a-b) of first and second adjustable arm supports (118a-b) may detect incomplete or improper placement of stowage cover (120).

Upon a determination that stowage cover (120) is not in a fully covering configuration, method (310) may proceed to step (318). At step (318), method (310) includes instructing a user to reposition stowage cover (120) over robotic arm (116) of table-based robotic system (110) in response to determining stowage cover (120) is in the non-covered configuration. In some versions, method (310) includes controller (192) providing at least one of an audible or visual alert to a user in response to the presence of first sensor target (176, 184) and the absence of a second sensor target (178, 186). Additionally, first sensor (194, 202) may continue to sense first sensor target (176, 184) to determine if stowage cover (120) becomes suitably positioned on table-based robotic system (110). The foregoing steps (314, 316, 318) may be reiterated until the user succeeds in positioning stowage cover (120) in a fully covering configuration on table-based robotic system (110).

In scenarios where applicable sensor targets (176, 178, 184, 188) are sensed at step (314), method proceeds to step (320). At step (320), method (310) includes generating applicable signals (222, 224, 226, 228) from applicable sensors (194, 196, 202, 204) in response to sensing the presence of applicable sensor targets (176, 178, 184, 188) of stowage cover (120) via applicable sensors (194, 196, 202, 204) of table-based robotic system (110). Next, at step (322), method (310) includes transmitting applicable signals (222, 224, 226, 228) from applicable sensors (194, 196, 202, 204) to controller (192).

At step (324), method (310) includes determining, via controller (192), that stowage cover (120) is in the covered configuration based on the signal(s) signals (222, 224, 226, 228). For two or more positions sensors, controller (196) may determine that stowage cover (120) is in a covered configuration based on the combination of signals. When the user correctly places stowage cover (120) over table-based robotic system (110), controller (192) detects the presence of stowage cover (120) and communicates to table-based robotic system (110) that stowage cover (120) is present.

At step (326), method (310) includes preventing movement of the at least one movable robotic arm (116) via controller (192) in response to determining that stowage cover (120) is in the covered configuration. For example, when the user at tower (220) attempts to move robotic arms (116), controller (192) prevents movement of robotic arms (116). First and second bars (136a-b) may sense the presence of sensor targets (176, 178, 184, 186) (e.g., magnets) at any of the four bar locations (e.g., first and second ends (198, 200, 206, 208)) via the triggered sensor (194, 196, 202, 204) (e.g., Hall effect sensor).

At step (328), an instruction or alert may be provided to the user. For example, an error message on tower (220) may prompt the user to confirm when stowage cover (120) has been removed. In some versions, controller (192) may continue to prevent operation of robotic arms (116) until the alert is manually cleared by the user at step (330). In some versions, the tower (220) may emit an error signal and user may clear the error signal or otherwise fix the issue. For example, the user may seek to remove one of first and second flexible bodies (140a-b) while leaving the other of first and second flexible bodies (140a-b) in the covered configuration.

At step (332), method (310) includes the user removing stowage cover (120) from table-based robotic system (110). First and second flexible bodies (140a-b) of stowage cover (120) may fold up when stowage cover (120) is not disposed on table-based robotic system (110), so that a user may fold up and store stowage cover (120) in the operating room. For example, stowage cover (120) may be stored in tower (220) or elsewhere. In the folded state, stowage cover (120) occupies less volume than in an unfolded state.

As a result of removing stowage cover (120), sensors (194, 196, 202, 204) no longer detect the presence of stowage cover (120) and communicate to table-based robotic system (110) that stowage cover (120) is absent. Controller (192) may then allow the user to operate robotic arms (116) in the normal course during a robotic surgical procedure, etc. After the robotic surgical procedure is complete, controller (192) may activate an alert to remind the user to replace stowage cover (120).

In some versions, when the user attempts to physically move table-based robotic system (110) (e.g., from one room to another room after completion of a robotic surgical procedure), controller (192) checks for signals (222, 224, 226, 228) from sensors (194, 196, 202, 204) for presence for stowage cover (120). Controller (192) may notify the user to place stowage cover (120) on prior to physically moving table-based robotic system (110). In some such versions, controller (192) may prevent movement of table-based robotic system (110) until stowage cover (120) is detected. For instance, controller (192) may activate brakes or other locking mechanisms on wheels of support structure (112) until stowage cover (120) is detected.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A robotic surgical system comprising: (a) a plurality of robotic arms including first and second independently movable robotic arms, wherein the plurality of robotic arms is operable to transition between a stowed configuration and a deployed configuration; (b) an arm support operatively coupled with the plurality of robotic arms; and (c) a stowage cover that includes a flexible body defining a first compartment and a second compartment, wherein the first compartment is configured to simultaneously cover the first and second independently movable robotic arms in the stowed configuration and the second compartment is configured to cover the arm support in the stowed configuration.

### Example 2

The robotic surgical system of Example 1, further comprising a surgical bed including a table configured to receive a patient, wherein the arm support is further coupled with the surgical bed, wherein the first and second independently movable robotic arms are positioned under the table in the stowed configuration, wherein the first and second independently movable robotic arms are configured to move relative to the table in the deployed configuration.

The robotic surgical system of Example 2, wherein the plurality of robotic arms further comprises a third independently movable robotic arm, wherein the first, second, and third independently movable robotic arms are positioned under the table in the stowed configuration, wherein the first, second, and third independently movable robotic arms are configured to move relative to the table in the deployed configuration, wherein the first compartment is configured to simultaneously cover the first, second, and third independently movable robotic arms in the stowed configuration.

### Example 3

### Example 4

The robotic surgical system of any of Examples 2 through 3, wherein the arm support includes a bar that extends parallel to the table.

### Example 5

The robotic surgical system of any of Examples 1 through 4, wherein the first compartment of the stowage cover is configured to closely conform to the plurality of robotic arms.

### Example 6

The robotic surgical system of any of Examples 1 through 5, wherein the stowage cover includes at least one stiffening member configured to provide rigidity to a predetermined portion of the flexible body.

### Example 7

The robotic surgical system of any of Examples 1 through 6, wherein the first compartment of the stowage cover includes indicia indicating surfaces to facilitate positioning of the stowage cover over the first and second independently movable robotic arms.

### Example 8

The robotic surgical system of Example 7, further comprising a second arm support, wherein the plurality of robotic arms includes third and fourth independently movable robotic arms operatively coupled with the second arm support, wherein the stowage cover further comprises a second flexible body defining a first compartment and a second compartment, wherein the first compartment of the second flexible body is configured to simultaneously cover the third and fourth independently movable robotic arms in the stowed configuration and the second compartment of the second flexible body is configured to cover the second arm support in the stowed configuration.

### Example 9

The robotic surgical system of any of Examples 1 through 8, wherein the stowage cover includes at least one pull tab configured to interacted with by a user to position the stowage cover relative to the first and second independently movable robotic arms.

### Example 10

The robotic surgical system of any of Examples 1 through 9, wherein the first and second compartments are joined together at seams.

### Example 11

The robotic surgical system of any of Examples 1 through 10, wherein the flexible body includes a liquid-impermeable material.

### Example 12

The robotic surgical system of any of Examples 1 through 11, wherein the stowage cover includes a first sensor target operatively coupled with the flexible body, the robotic surgical system further comprising: (a) a first sensor configured to generate a first signal in response to the first sensor sensing a presence of the first sensor target; and (b) a controller configured to receive the first signal from the first sensor to determine that the stowage cover is positioned on the first and second independently movable robotic arms.

### Example 13

The robotic surgical system of Example 12, wherein the controller is configured to prevent the first and second independently movable robotic arms from transitioning to the deployed configuration in response to determining that the stowage cover is in the covered configuration.

### Example 14

The robotic surgical system of any of Examples 12 through 13, wherein the first sensor includes a proximity sensor, wherein the first sensor target includes a proximity sensor target configured to be sensed by the proximity sensor.

### Example 15

The robotic surgical system of any of Examples 12 through 13, wherein the first sensor includes a Hall effect sensor, wherein the first sensor target includes a magnet configured to be sensed by the Hall effect sensor.

### Example 16

The robotic surgical system of Example 15, wherein the arm support includes a ferromagnetic feature configured to magnetically couple with the magnet to couple the stowage cover with the arm support.

### Example 17

A method of operating a robotic surgical system that includes first and second independently movable robotic arms and an arm support operatively coupled with the first and second independently movable robotic arms, the method comprising: (a) receiving a first compartment of a stowage cover over the first and second independently movable robotic arms when the first and second independently movable robotic arms are in a stowed configuration; and (b) receiving the arm support in a second compartment of the stowage cover.

### Example 18

The method of Example 17, further comprising moving the arm support between an upper position and a lower position while the first compartment of the stowage cover is positioned over the first and second independently movable robotic arms and the second compartment is positioned over the arm support.

### Example 19

The method of Example 18, wherein the act of receiving the arm support further comprises positioning the stowage cover over the arm support and the first and second independently movable robotic arms when the first and second independently movable robotic arms are in the stowed configuration, and the act of moving further comprises moving the arm support between the upper position and the lower position while the stowage cover is positioned over the arm support and the first and second independently movable robotic arms.

### Example 20

The method of any of Examples 17 through 19, wherein the acts of receiving the first compartment of the stowage cover over the first and second independently movable robotic arms and receiving the arm support in the second compartment of the stowage cover comprise positioning the stowage cover under a table of a surgical bed of the robotic surgical system, wherein the table is configured to receive a patient.

### Example 21

The method of any of Examples 17 through 20, further comprising performing a manual procedure on the patient while the patient is on the surgical bed while the first and second independently movable robotic arms and the stowage cover are positioned under the surgical bed.

### Example 22

The method of any of Examples 17 through 21, the robotic surgical system further comprising a third independently movable robotic arm, wherein in the stowed configuration, the first, second, and third independently movable robotic arms are positioned under the table.

### Example 23

The method of any of Examples 17 through 22, further comprising, in response to the act of receiving the first compartment of the stowage cover over the first and second independently movable robotic arms, preventing movement of the first and second independently movable robotic arms using a controller.

### Example 24

The method of any of Examples 17 through 23, further comprising: (a) sensing a presence of a first sensor target of the stowage cover via a first sensor of the robotic surgical system; and (b) generating a first signal from the first sensor, the first signal indicating the sensing of the presence of the first sensor target.

### Example 25

The method of Example 24, wherein the arm support includes a bar, wherein the bar includes the first sensor.

### Example 26

A robotic surgical system comprising: (a) a surgical bed comprising a table configured to receive a patient; (b) a first movable robotic arm coupled with the surgical bed and movable between a stowed configuration and a deployed configuration, wherein the first movable robotic arm is positioned under the table in the stowed configuration and is configured to interact with the patient in the deployed configuration; (c) a stowage cover comprising: (i) a flexible body configured to selectively cover the first movable robotic arm in the stowed configuration, wherein the flexible body is removable from the surgical bed to permit the first movable robotic arm to transition from the stowed configuration to the deployed configuration, and (ii) a first sensor target operatively coupled with the flexible body; (d) a first sensor configured to generate a first signal in response to sensing a presence of the first sensor target; and (e) a controller configured to receive the first signal from the first sensor to determine that the stowage cover is positioned on the first movable robotic arm in the stowed configuration.

### Example 27

The robotic surgical system of Example 26, wherein, in response to the first sensor not sensing the presence of the first sensor target, the controller is configured to provide an alert to a user.

### Example 28

The robotic surgical system of any of Examples 26 through 27, wherein the first sensor includes a proximity sensor, wherein the first sensor target includes a proximity sensor target configured to be sensed by the proximity sensor.

### Example 29

The robotic surgical system of Example 28, wherein the proximity sensor includes a Hall effect sensor, wherein the proximity sensor target includes a magnet configured to be sensed by the Hall effect sensor.

### Example 30

The robotic surgical system of Example 29, further comprising an arm support operatively coupled with the first movable robotic arm, wherein the arm support includes a magnetic feature configured to magnetically couple with the magnet to couple the stowage cover with the arm support.

### Example 31

The robotic surgical system of any of Examples 26 through 30, further comprising a second sensor, wherein the stowage cover includes a second sensor target operatively coupled with the flexible body, wherein the second sensor is configured to sense a presence of the second sensor target, wherein the controller is configured to determine that the stowage cover is positioned on the first movable robotic arm based on a combination of the first sensor sensing the presence of the first sensor target and the second sensor sensing the presence of the second sensor target.

### Example 32

The robotic surgical system of Example 31, wherein, in response to either the first sensor not sensing the presence of the first sensor target or the second sensor not sensing the presence of the second sensor target, the controller is configured to provide an alert to a user.

### Example 33

The robotic surgical system of any of Examples 31 through 32, wherein the first sensor includes a first Hall effect sensor, wherein the second sensor includes a second Hall effect sensor, wherein the first sensor target includes a first magnet, wherein the second sensor target includes a second magnet, wherein the controller is configured to determine that the stowage cover is positioned on the first movable robotic arm based on a combination of the first Hall effect sensor sensing the presence of the first magnet and the second Hall effect sensor sensing the presence of the second magnet.

### Example 34

The robotic surgical system of Example 33, wherein the flexible body includes opposing first and second ends, wherein the first magnet is positioned at the first end, wherein the second magnet is positioned at the second end.

### Example 35

The robotic surgical system of any of Examples 33 through 34, wherein the arm support includes opposing first and second magnetic features configured to magnetically couple with the first and second magnets to couple the stowage cover with the base.

### Example 36

The robotic surgical system of any of Examples 26 through 35, wherein the stowage cover includes at least one pull tab configured to be interacted with by a user to position the cover relative to the first movable robotic arm.

### Example 37

The robotic surgical system of any of Examples 26 through 36, wherein the stowage cover includes at least one stiffening member configured to provide rigidity to a predetermined portion of the flexible body.

### Example 38

The robotic surgical system of any of Examples 26 through 37, further comprising a second movable robotic arm that is independently movable relative to the first movable robotic arm, wherein the stowage cover is configured to cover each of the first and second movable robotic arms simultaneously.

### Example 39

The robotic surgical system of any of Example 38, wherein the flexible body defines a first compartment and a second compartment, wherein the first compartment is configured to simultaneously cover the first and second movable robotic arms in the stowed configuration and the second compartment is configured to cover the arm support in the stowed configuration.

### Example 40

The robotic surgical system of any of Examples 26 through 39, wherein the flexible body includes a liquid-impermeable material.

### Example 41

The robotic surgical system of any of Examples 39 through 40, wherein the first and second movable robotic arms are operable to transition between a stowed configuration and a deployed configuration, wherein the stowage cover is configured to cover the first and second movable robotic arms in at least the stowed configuration.

### Example 42

The robotic surgical system of any of Examples 40 through 41, further comprising a bar that is coupled with the first and second movable robotic arms, wherein the bar is configured to transition between an upper position and a lower position, wherein the stowage cover is configured to cover the first and second movable robotic arms and the bar in the upper position, in the lower position, and during the transition between the upper position and the lower position.

### Example 43

The robotic surgical system of any of Examples 38 through 42, wherein the flexible body includes indicia indicating surfaces to facilitate positioning of the stowage cover over the first and second movable robotic arms.

### Example 44

The robotic surgical system of Example 43, wherein the indicia are positioned on each of the first and second compartments and are configured to aid a user in installing of the stowage cover over the first and second movable robotic arms.

A method of operating a robotic surgical system, the method comprising: (a) receiving a stowage cover over at least one movable robotic arm of the robotic surgical system; (b) sensing a presence of a first sensor target of the stowage cover via a first sensor of the robotic surgical system; and (c) generating a first signal from the first sensor, the first signal indicating the sensing of the presence of the first sensor target.

### Example 45

### Example 46

The method of Example 45, further comprising: (a) transmitting the first signal from the first sensor to a controller; and (b) determining, via the controller, that the stowage cover is in a covered configuration based on the first signal.

The method of Example 46, further comprising, alerting the user to remove the stowage cover from the at least one movable arm in response to both determining that the stowage cover is in the covered configuration and a receiving a signal to the robotic surgical system to move the at least one movable robotic arm.

### Example 47

### Example 48

The method of Example 45, further comprising sensing an absence of the first sensor target of the stowage cover via the first sensor of the robotic surgical system, the absence being sensed in response to the stowage cover being removed from the at least one movable robotic arm of the robotic surgical system.

### Example 49

The method of Example 48, further comprising: (a) in response to sensing the absence of the first sensor target, transmitting a second signal from the first sensor to a controller; and (b) determining, via the controller, that the stowage cover is in a non-covered configuration based on the first signal from the first sensor.

### Example 50

The method of Example 49, further comprising, in response to determining that the stowage cover is in the covered configuration, preventing movement of the at least one movable robotic arm via the controller.

### Example 51

The method of any of Examples 47 through 50, further comprising, in response to sensing the absence of the first sensor target, instructing a user to reposition the stowage cover over the at least one movable arm of the robotic surgical system.

### Example 52

The method of any of Examples 45 through 50, wherein the first sensor includes a first Hall effect sensor, wherein the first sensor target includes a first magnet, wherein the act of sensing further comprises sensing the presence of the first magnet of the stowage cover via the first Hall effect sensor.

### Example 53

The method of any of Examples 45 through 52, further comprising: (a) sensing a presence of a second sensor target of the stowage cover via a second sensor of the robotic surgical system; and (b) generating a second signal from the second sensor, the second signal indicating the sensing of the presence of the second sensor target.

### Example 54

The method of Example 53, further comprising: (a) transmitting the first signal from the first sensor to a controller; (b) transmitting the second signal from the second sensor to the controller; and (c) determining, via the controller, that the stowage cover is in a covered configuration based on the combination of the first and second signals.

### Example 55

The method of Example 54, wherein the first sensor includes a first Hall effect sensor, wherein the second sensor includes a second Hall effect sensor, wherein the first sensor target includes a first magnet, wherein the second sensor target includes a second magnet, the acts of sensing further comprising: (i) sensing the presence of the first magnet of the stowage cover via the first Hall effect sensor, and (ii) sensing the presence of the second magnet of the stowage cover via the second Hall effect sensor.

### Example 56

The method of any of Example 45, further comprising: (a) sensing an absence of a second sensor target of the stowage cover via a second sensor of the robotic surgical system; and (b) providing an alert to a user in response to the presence of first sensor target and the absence of the second sensor target.

### Example 57

The method of Example 56, further comprising locking out operation of the at least one movable robotic arm until the alert is manually cleared by the user.

### Example 58

The method of any of Examples 45 through 57, wherein the act of positioning the stowage cover further comprises positioning the stowage cover under a table of the robotic surgical system, wherein the table is configured to receive a patient.

### Example 59

A stowage cover comprising: (a) a flexible body configured to cover a portion of a robotic surgical system, wherein the body includes opposing first and second ends; (b) at least one stiffening member configured to provide rigidity to a predetermined portion of the flexible body; (c) a first magnet disposed at the first end and configured to be sensed by a first Hall effect sensor of the robotic surgical system; and (d) a second magnet disposed at the second end and configured to be sensed by a second Hall effect sensor of the robotic surgical system.

### Example 60

The stowage cover of Example 59, further comprising at least one pull tab configured to interacted with by a user to position the stowage cover relative to the at least one movable robotic arm.

### IV. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A robotic surgical system comprising:
(a) a surgical bed comprising a table configured to receive a patient;
(b) a first movable robotic arm coupled with the surgical bed and movable between a stowed configuration and a deployed configuration, wherein the first movable robotic arm is positioned under the table in the stowed configuration and is configured to interact with the patient in the deployed configuration;
(c) a stowage cover comprising:
(i) a flexible body configured to selectively cover the first movable robotic arm in the stowed configuration, wherein the flexible body is removable from the surgical bed to permit the first movable robotic arm to transition from the stowed configuration to the deployed configuration, and
(ii) a first sensor target operatively coupled with the flexible body;
(d) a first sensor configured to generate a first signal in response to sensing a presence of the first sensor target; and
(e) a controller configured to receive the first signal from the first sensor to determine that the stowage cover is positioned on the first movable robotic arm in the stowed configuration.

2. The robotic surgical system of claim 1, wherein, in response to the first sensor not sensing the presence of the first sensor target, the controller is configured to provide an alert to a user.

3. The robotic surgical system claim 1, wherein:
the first sensor includes a proximity sensor, the proximity sensor including a Hall effect sensor; and
the first sensor target includes a proximity sensor target configured to be sensed by the proximity sensor, the proximity sensor target including a magnet configured to be sensed by the Hall effect sensor.

4. The robotic surgical system of claim 3, further comprising an arm support operatively coupled with the first movable robotic arm, wherein the arm support includes a magnetic feature configured to magnetically couple with the magnet to couple the stowage cover with the arm support.

5. The robotic surgical system of claim 1, further comprising a second sensor, wherein:
the stowage cover includes a second sensor target operatively coupled with the flexible body,
the second sensor is configured to sense a presence of the second sensor target, and
the controller is configured to determine that the stowage cover is positioned on the first movable robotic arm based on a combination of the first sensor sensing the presence of the first sensor target and the second sensor sensing the presence of the second sensor target.

6. The robotic surgical system of claim 5, wherein:
the first sensor includes a first Hall effect sensor,
the second sensor includes a second Hall effect sensor,
the first sensor target includes a first magnet,
the second sensor target includes a second magnet, and
the controller is configured to:
determine that the stowage cover is positioned on the first movable robotic arm based on a combination of the first Hall effect sensor sensing the presence of the first magnet and the second Hall effect sensor sensing the presence of the second magnet, and
in response to either the first sensor not sensing the presence of the first sensor target or the second sensor not sensing the presence of the second sensor target, the controller is configured to provide an alert to a user.

7. The robotic surgical system of claim 6, wherein the flexible body includes opposing first and second ends, wherein the first magnet is positioned at the first end, wherein the second magnet is positioned at the second end, and wherein an arm support operatively coupled with the first movable robotic arm includes opposing first and second magnetic features configured to magnetically couple with the first and second magnets to couple the stowage cover with the arm support.

8. The robotic surgical system of claim 1, further comprising a second movable robotic arm that is independently movable relative to the first movable robotic arm, wherein the stowage cover is configured to cover each of the first and second movable robotic arms simultaneously.

9. The robotic surgical system of claim 8, wherein:
the flexible body defines a first compartment and a second compartment,
the first compartment is configured to simultaneously cover the first and second movable robotic arms in the stowed configuration, and
the second compartment is configured to cover an arm support operatively coupled with the first and second movable robotic arms in the stowed configuration.

10. The robotic surgical system of claim 1, wherein the flexible body includes a liquid-impermeable material.

11. The robotic surgical system of claim 8, wherein:
the first and second movable robotic arms are operable to transition between a stowed configuration and a deployed configuration, and
the stowage cover is configured to cover the first and second movable robotic arms in at least the stowed configuration.

12. The robotic surgical system of claim 8, further comprising a bar that is coupled with the first and second movable robotic arms, wherein the bar is configured to transition between an upper position and a lower position, and wherein the stowage cover is configured to cover the first and second movable robotic arms and the bar in the upper position, in the lower position, and during the transition between the upper position and the lower position.

13. The robotic surgical system of claim 9, wherein the flexible body includes indicia indicating surfaces to facilitate positioning of the stowage cover over the first and second movable robotic arms, and wherein the indicia are positioned on each of the first and second compartments and are configured to aid a user in installing of the stowage cover over the first and second movable robotic arms.

14. A method of operating a robotic surgical system, the method comprising:
(a) receiving a stowage cover over at least one movable robotic arm of the robotic surgical system;
(b) sensing a presence of a first sensor target of the stowage cover via a first sensor of the robotic surgical system; and
(c) generating a first signal from the first sensor, the first signal indicating the sensing of the presence of the first sensor target.

15. The method of claim 14, further comprising:
(a) transmitting the first signal from the first sensor to a controller; and
(b) determining, via the controller, that the stowage cover is in a covered configuration based on the first signal.

16. The method of claim 15, further comprising, alerting a user to remove the stowage cover from the at least one movable robotic arm in response to both determining that the stowage cover is in the covered configuration and a receiving a signal to the robotic surgical system to move the at least one movable robotic arm.

17. The method of claim 15, further comprising:
(a) sensing an absence of the first sensor target of the stowage cover via the first sensor of the robotic surgical system, the absence being sensed in response to the stowage cover being removed from the at least one movable robotic arm of the robotic surgical system;
(b) in response to sensing the absence of the first sensor target, transmitting a second signal from the first sensor to a controller;
(c) determining, via the controller, that the stowage cover is in a non-covered configuration based on the first signal from the first sensor; and
(d) in response to determining that the stowage cover is in the covered configuration, preventing movement of the at least one movable robotic arm via the controller.

18. The method of claim 17, further comprising, in response to sensing the absence of the first sensor target, instructing a user to reposition the stowage cover over the at least one movable robotic arm of the robotic surgical system.

19. The method of claim 15, wherein:
the first sensor includes a first Hall effect sensor,
the first sensor target includes a first magnet, and
the act of sensing further comprises sensing the presence of the first magnet of the stowage cover via the first Hall effect sensor.

20. The method of claim 15, wherein the act of positioning the stowage cover further comprises positioning the stowage cover under a table of the robotic surgical system, wherein the table is configured to receive a patient.
